(19) United States
(12) Patent Application Publication
(10) Pub. No.: US 2024/0026301 A1
(43) Pub. Date: Jan. 25, 2024

(54) METHODS AND COMPOSITIONS FOR GENERATING ENDOTHELIAL CELLS FROM PLURIPOTENT STEM CELLS

(71) Applicant: Trailhead Biosystems Inc., Cleveland, OH (US)

(72) Inventor: Angelica M. GOMES UELTSCHY, Shaker Heights, OH (US)

(21) Appl. No.: 18/224,696

(22) Filed: Jul. 21, 2023

Related U.S. Application Data
(60) Provisional application No. 63/391,208, filed on Jul. 21, 2022.

(51) Int. Cl. C12N 5/071

(52) U.S. Cl. CPC C12N 5/069; C12N 2501/165; C12N 2501/41; C12N 2501/415; C12N 2501/91; C12N 2501/115; C12N 2506/45

(57) ABSTRACT

Methods for generating CD31+VE−cadherin+KDR+ endothelial cells (ECs) are provided using chemically-defined culture media that allow for generating ECs from early mesoderm progenitors in as little as three days and from pluripotent stem cells in as little as five days. Culture media, isolated cell populations and kits are also provided.

### RELATED APPLICATION

This application claims priority to U.S. Provisional Application No. 63/391,208, filed Jul. 21, 2022, the entire contents of which is hereby incorporated by reference.

### BACKGROUND OF THE INVENTION

Endothelial cells (ECs) are the main type of cells found in the lining of the blood vessels, lymph vessels and the heart. Damage to, or loss of, blood vessels is the main pathophysiological feature of vascular diseases, including ischemic cardiovascular diseases. Treatment of such diseases remains challenging, despite considerable effort being made. Given the key role of ECs in the pathophysiology, approaches that focus on ECs or growing blood vessels make sense biologically. The ability to generate endothelial cells from stem cell, such as pluripotent stem cells, thus is important both for vascular research and for revascularization. For example, readily available stem cell-derived ECs would allow for their potential use in inducing therapeutic neovascularization, enhancing proper blood perfusion or promoting tissue repair (see e.g., Losordo et al. (2004) Circulation 109:2692-2697). For an overview of ECs derived from pluripotent stem cells, see e.g., Jang et al. (2019) Am. J. Pathol. 189:502-512. Moreover, iPSC-derived endothelial cells can be used for drug discovery, either for identification of new anti-angiogenic drugs, or for safety assessment in the context of drug-induced vascular toxicity.

The earliest methodologies for generating ECs from pluripotent stem cells devised three different approaches (as reviewed in, for example, Wilson et al. (2014) Stem Cells 32:3037-3045; Yoder (2015) Curr. Opin. Hematol. 22:252-257; Williams and Wu (2019) Arter. Thromb. Vasc. Biol. 39:1317-1329). In the first approach, embryonic stem cells (ESCs) or induced pluripotent stem cells (iPSCs) are grown under conditions that induce self-aggregation of the cells into embryoid bodies (EBs) and then a variety of growth factors are used to promote EC differentiation with the EBs. In the second approach, differentiating ESCs or iPSCs are co-cultured with a feeder layer, such as stromal cells (e.g., murine calvarial mesenchymal OP9 cells or bone marrow stromal cells), to promote differentiation along the EC lineage. In the third approach, ESCs or iPSCs are grown in two-dimensional culture on plates coated with a protein substrate, such as Matrigel, fibronectin, vitronectin, gelatin or similar protein, under specific conditions with added growth factors.

A chemically-defined culture protocol for generating ECs from human pluripotent stem cells has been described involving modulation of canonical Wnt signaling (see e.g., Lian et al. (2014) Stem Cell Reports 3:804-816; Bao et al. (2015) Stem Cell Res. 15:122-129). For example, albumin-free culture conditions have been reported in which pluripotent stem cells were cultured in DMEM base media, supplemented with ascorbic acid, and treated with a GSK-inhibitor (CHIR99012), which activates Wnt signaling. This protocol was shown to be sufficient to generate CD34+CD31+ endothelial progenitors, although the yield of cells was low (e.g., 20-30% CD34+CD31+ cells prior to sorting).

Furthermore, the MAPK and PI3K pathways have been implicated in the differentiation of ECs from PSCs, with a culture protocol reported in which GSK-3β inhibitor treatment was followed by treatment with VEGF, FGF2 and BMP4. These growth factors were used to induce early vascular progenitors from hiPSC-derived mesodermal cells, leading to generation of CD31+VE−cadherin+ ECs in eight days (Harding et al. (2017) Stem Cells 35:909-919). Cyclic AMP has been reported to enhance the effects of VEGF (Ikuno et al. (2017) PLoS One 12:e0173271). A subsequent chemically-defined protocol also combined GSK-3β inhibitor treatment with culture conditions utilizing VEGF, FGF2 and BMP4, as well as adding DAPT and forskolin (Farkas et al. (2020) Front. Cell. Devel. Biol. 8:309).

Accordingly, while some progress has been, there remains a need for efficient and robust methods and compositions for generating endothelial cells from pluripotent stem cells in culture in large scale.

### SUMMARY OF THE INVENTION

This disclosure provides methods of generating endothelial cells (ECs) using chemically-defined culture media that allows for robust generation of CD31+ ECs from pluripotent stem cells in as little as five days of culture. The culture media comprises small molecule agents that either agonize or antagonize particular signaling pathways such that differentiation along the endothelial cell lineage is promoted. The methods of the disclosure have the advantage that they have a high % endothelial conversion (80-90%) and allow for robust endothelial cell generation, including using bioreactors, in a short time span using reagents costing less than current protocols. Further culture of the resultant ECs in endothelial cell media leads to cells that exhibit endothelial cell functionality, such as measured by a tube formation assay, an ac-LDL assay or a nitric oxide production assay. Additionally, the use of small molecule agents in the culture media allows for precise control of the culture components.

The methods and compositions of the disclosure utilize a unique cell culture media that allows for generation of CD31+ endothelial cells from early mesoderm progenitor cells in as little as three days. This unique cell culture media includes certain components previously used for endothelial cell generation, such as a VEGFR agonist and an FGFR agonist, but also includes novel components and combinations that allow for robust endothelial cell generation. Early mesoderm progenitor cells can be obtained by culturing pluripotent stem cells under culture conditions that generate the early mesoderm progenitors, as described herein. Thus, in certain embodiments, the methods comprise a single stage culture protocol, starting with early mesoderm progenitors and resulting in CD31+ endothelial cells in three days. In other embodiments, the methods comprise a two-stage culture protocol, starting with pluripotent stem cells being differentiated for two days into early mesoderm progenitors in stage 1 and then further differentiating the early mesoderm progenitors for three days into CD31+ endothelial cells as stage 2, resulting in a two-stage, five day protocol.

Accordingly, in one aspect, the disclosure pertains to a method of generating human CD31+ endothelial cells (ECs) comprising:
culturing human early mesoderm progenitor cells in a culture media comprising a VEGFR agonist, an FGFR agonist, a retinoic acid pathway agonist, a sonic hedgehog (SHH) antagonist, heparin or heparin mimetic and a WNT pathway antagonist to generate human CD31+ ECs.

In an embodiment, the early mesoderm progenitor cells are cultured for three days in the culture media to obtain CD31+ endothelial cells.

In an embodiment, the early mesoderm progenitor cells are obtained by culturing human pluripotent stem cells in a media comprising a Wnt pathway agonist for two days.

In another aspect, the disclosure pertains to a two stage method of generating human CD31+ endothelial cells (ECs), the method comprising:
(a) culturing human pluripotent stem cells in a culture media comprising a Wnt pathway agonist on day 0-day2 to generate early mesoderm progenitor cells; and
(b) culturing the early mesoderm progenitor cells in a culture media comprising a VEGFR agonist, an FGFR agonist, a retinoic acid pathway agonist, a sonic hedgehog (SHH) antagonist, heparin or heparin mimetic and a WNT pathway antagonist on day 2-day 5 to generate CD31+ ECs.

In certain embodiments, the CD31+ ECs generated by a method of the disclosure also express at least one marker selected from the group consisting of KDR, vWF, CD34, FL1, VE-cadherin and CD31. In certain embodiments, the CD31+ ECs express at least two, at least three, at least four, at least five or all six of the markers selected from the group consisting of KDR, vWF, FL1, VE−cadherin, CD34 and CD31.

In an embodiment, the Wnt pathway agonist used in the culture media for generating early mesoderm progenitors is a GSK-3β inhibitor. In an embodiment, the GSK-3β inhibitor is CHIR99021. In an embodiment, CHIR99021 is present in the culture at a concentration in a range of 3.0-9.0 μM. In an embodiment, CHIR99021 is present in the culture at a concentration of 6.0 μM.

In an embodiment, the VEGFR agonist is VEGF. In an embodiment, VEGF is present in the culture at a concentration in a range of 10-50 ng/ml. In an embodiment, VEGF is present in the culture at a concentration of 25 ng/ml.

In an embodiment, the FGFR agonist is FGF2 or SUN11602. In an embodiment, the FGFR agonist is present in the culture at a concentration in a range of 1-20 ng/ml. In an embodiment, the FGFR agonist is FGF2, which is present in the culture at a concentration in a range of 1-20 ng/ml. In an embodiment, the FGFR agonist is FGF2, which is present in the culture at a concentration of 10 ng/ml.

In an embodiment, the retinoic acid (RA) pathway agonist is selected from the group consisting of TTNPB, AM 580, CD 1530, CD 2314, CD 437, Ch 55, BMS 753, BMS 961, Tazarotene, Tamibarotene, Isotretinoin, Tretinoin, AC 261066, AC 55649, retinoic acid (RA), Sr11237, adapalene, EC23, 9-cis retinoic acid, 13-cis retinoic acid, 4-oxo retinoic acid, and All-trans Retinoic Acid (ATRA), AY 9944 dihydrochloride, Ciliobrevin A, Cyclopamine, or combinations thereof. In an embodiment, the RA pathway agonist is present in the culture at a concentration in a range of 10-100 nM. In an embodiment, the RA pathway agonist is TTNPB, which is present in the culture at a concentration in a range of 25-75 nM. In an embodiment, the RA pathway agonist is TTNPB, which is present in the culture at a concentration of 50 nM.

In an embodiment, the sonic hedgehog (SHH) antagonist is selected from the group consisting of Sant-1, Saikosaponin Bl, Itraconazole, GANT61, MK4101, HPI-4, Vismodegib, Jervine, JK184, Taladegib, Ciliobrevin D, Dynapyrazole A, Dynarrestin, GANT58, HPI1, IHR1, PF 04449913 maleate, SANT-2, U 18666A, and combinations thereof. In an embodiment, the SHH antagonist is present in the culture at a concentration in a range of 10-100 nM. In an embodiment, the SHH antagonist is Sant-1, which is present in the culture at a concentration in a range of 25-75 nM. In an embodiment, the SHH antagonist is Sant-1, which is present in the culture at a concentration of 50 nM.

In an embodiment, heparin or a heparin mimetic is selected from the group consisting of heparin, heparan sulfate, enoxaparin, small molecular weight heparins, AV5026, M402, and combinations thereof. In an embodiment, heparin or a heparin mimetic is present in the culture at a concentration in a range of 10-50 ng/ml. In an embodiment, heparin is present in the culture at a concentration in a range of 25-35 ng/ml. In an embodiment, heparin is present in the culture at a concentration of 20 ng/ml.

In an embodiment, the Wnt pathway antagonist is selected from the group consisting of XAV939, ICG-001 (Foscenvivint), Capmatinib (INCB28060), endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, iCRT14, WIF-1, DKK1, Isoquercitrin, Lanatoside C, Gigantol, RCM-1, WIKI4, IQ-1, Adavivant, PRI-724, Tegatrabetan, or combinations thereof. In an embodiment, the Wnt pathway antagonist is present in the culture at a concentration in a range of 10-500 nM. In an embodiment, the Wnt pathway antagonist is XAV939, which is present in the culture at a concentration in a range of nM. In an embodiment, the Wnt pathway antagonist is XAV939, which is present in the culture at a concentration of 100 nM.

In an embodiment, the pluripotent stem cells are embryonic stem cells. In an embodiment, the pluripotent stem cells are induced pluripotent stem cells.

In another aspect, the disclosure pertains to a culture media for generating endothelial cells (ECs) comprising a VEGFR agonist, an FGFR agonist, a retinoic acid (RA) pathway agonist, a sonic hedgehog (SHH) antagonist, heparin or heparin mimetic and a WNT pathway antagonist. Suitable agonists and antagonists, and concentration ranges, are described herein. In an embodiment, the VEGFR agonist is VEGF, the FGFR agonist is FGF2, the RA pathway agonist is TTNBP, the SHH antagonist is Sant-1, the heparin or heparin mimetic is heparin and the WNT pathway antagonist is XAV939. In an embodiment, VEGF is at a concentration of 25 ng/ml, FGF2 is at a concentration of 10 ng/ml, TTNBP is at a concentration of 50 nM, Sant-1 is at a concentration of 50 nM, heparin is at a concentration of 20 ng/ml and XAV939 is at a concentration of 100 nM.

In another aspect, the disclosure pertains to an isolated cell culture of human CD31+ endothelial cells, the culture comprising: human CD31+ endothelial cells cultured in a culture media comprising a VEGFR agonist, an FGFR agonist, a retinoic acid (RA) pathway agonist, a sonic hedgehog (SHH) antagonist, heparin or heparin mimetic and a WNT pathway antagonist. Suitable agonists and antagonists, and concentration ranges, are described herein.

Other features and advantages of the invention will be apparent from the following detailed description and claims.

### BRIEF DESCRIPTION OF THE DRAWINGS

FIG. 1 is a schematic diagram of a representative culture protocol for generating endothelial cells from early mesoderm progenitors in three days, as well as the two-day pre-treatment to generate the early mesoderm progenitors from iPSCs.

FIG. 2 shows the results from a model of an 8-factor experiment optimized for maximum expression of PECAM1 (CD31). The upper section of the model shows the prediction of expression level of pre-selected 53 genes when optimized for PECAM1. The lower section of the model shows the effectors that were tested in this model and their contribution to maximum expression of PECAM1. The value column refers to required concentration of each effector to mimic the model.

FIG. 3 shows the results from a model of an 8-factor experiment optimized for maximum expression of PECAM1 (CD31). Upper and lower sections are as described in FIG. 1.

FIG. 4 shows the results from a model of an 8-factor experiment optimized for maximum expression of PECAM1 (CD31). Upper and lower sections are as described in FIG. 1.

FIG. 5A-5B show the dynamic profile analysis of expression levels of PECAM1, CD34, CDH5 and FLI1 relative to the concentration of 3 validated effectors. The positive impact of VEGF, TTNBP and heparin on expression of genes above of and their factor contributions are shown by slope of the plots for each effector.

FIG. 6A-6B show the dynamic profile analysis of expression levels of PECAM1, CD34, CDH5 and Fli1 relative to the concentration of 3 validated effectors. The positive impact of VEGF on expression of all genes above of and their factor contributions are shown by slope of the plots for each effector. The positive impact of FGF2 on PECAM1, FLI1, and CDH5 expression. The positive impact of XAV939 on PECAM1 and Fli1 expression.

FIG. 7A-7B shows the dynamic profile analysis of expression levels of PECAM1, CD34, CDH5 and Fli1 relative to the concentration of 2 validated effectors. The positive impact of VEGF on expression of all genes above of and their factor contributions are shown by slope of the plots for each effector. The positive impact of SANT1 on PECAM1 and CDH5 expression.

FIG. 8 shows results of flow cytometry staining of iPSC-derived endothelial cells at the end of stage 2. Cells were stained with CD31, CD34, CD73, KDR, CXCR4 and CD144. At this stage, cells were positive for all the markers expected for endothelial cells.

FIG. 9 shows photographs of fluorescence images of iPSC-derived endothelial cells at the end of stage 2. Cells are stained with endothelial biomarkers including vWF, VE−cadherin, FLI1, KDR and Ulex europaeus Agglutinin I-FITC. At this stage, cells were positive for all the markers expected for endothelial cells.

FIG. 10A-10C show the results of experiments demonstrating functional validation of iPSC-derived endothelial cells after cryopreservation. FIG. 10A shows the results of a tube formation assay in a Matrigel layer after 3 h. FIG. 10B shows the results of acetylated LDL uptake by endothelial cells after 4 h of incubation, cells were stained with phalloidin (F-actin in white) as well. FIG. 10C shows the results of flow cytometry analysis of cells stained with DAF-FM, undifferentiated iPSC (red) were compared with iPSC derived endothelial cells (blue).

FIG. 11 shows the results of RNA-seq experiments used to characterize the iPSC-derived endothelial cells. Stage 1, stage 2, and cryopreserved stage 2 samples maintained for 4 days in culture were analyzed. Results are shown as a heat map of log 2 fold change showing differential gene expression of stage 2 cells compared to stage 1 cells (day 2). Blue represents genes downregulated in endothelial cells and red represents genes upregulated in endothelial cells.

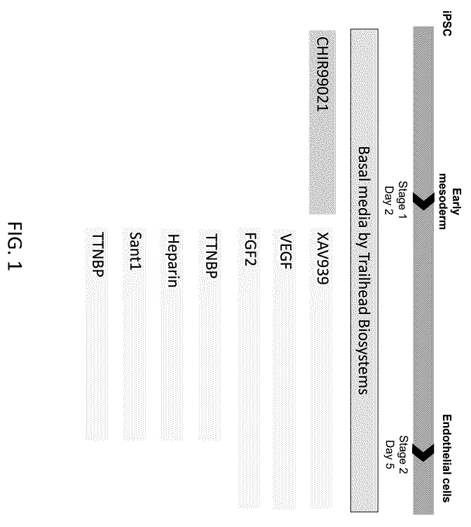

### DETAILED DESCRIPTION OF THE INVENTION

Described herein are methodologies and compositions that allow for the robust generation of KDR+CD31+VE−cadherin+ endothelial cells from iPSCs in only five days under chemically-defined culture conditions using a small molecule based approach. A High-Dimensional Design of Experiments (HD-DoE) approach was used to simultaneously test multiple process inputs (e.g., small molecule agonists or antagonists) on output responses, such as gene expression. These experiments allowed for the identification of chemically-defined culture media, comprising agonists and/or antagonists of particular signaling pathways, that is sufficient to generate ECs from iPSCs in a very short amount of time. The optimized culture media was further validated by a factor criticality analysis, which examined the effects of eliminating individual agonist or antagonist agents. Flow cytometry analysis and immunofluorescence was used to further confirmed the phenotype of the cells generated by the differentiation protocol.

Various aspects of the invention are described in further detail in the following subsections.

### I. Cells

The starting cells used in the cultures of the disclosure typically are human pluripotent stem cells, which are used to generate early mesoderm progenitors that are then used to obtain endothelial cells. As used herein, the term “human pluripotent stem cell” (abbreviated as hPSC) refers to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, for example, using a nude mouse and teratomas formation assay. Pluripotency can also evidenced by the expression of embryonic stem (ES) cell markers, although the preferred test for pluripotency is the demonstration of the capacity to differentiate into cells of each of the three germ layers.

Human pluripotent stem cells include, for example, induced pluripotent stem cells (iPSC) and human embryonic stem cells, such as ES cell lines. Non-limiting examples of induced pluripotent stem cells (iPSC) include 19-11-1, 19-9-7 or 6-9-9 cells (e.g., as described in Yu, J. et al. (2009) Science 324:797-801). Non-limiting examples of human embryonic stem cell lines include ES03 cells (WiCell Research Institute) and H9 cells (Thomson, J. A. et al. (1998) Science 282:1145-1147). Human pluripotent stem cells (PSCs) express cellular markers that can be used to identify cells as being PSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2. Since the methods of generating committed endothelial of the disclosure are used to differentiate (maturate) the starting pluripotent stem cell population, the resultant differentiated cells may lack expression of pluripotent stem cell markers. Accordingly, in various embodiments, the endothelial-committed cell populations generated by the methods of the disclosure lack expression of one or more stem cell markers, such as one or more stem cell markers selected from the group consisting of TRA-1-60, TRA-1-81, TRA-2-54, SSEA1, SSEA3, SSEA4, CD9, CD24, OCT3, OCT4, NANOG and/or SOX2.

The pluripotent stem cells are subjected to culture conditions, as described herein, that induce cellular differentiation. As used herein, the term “differentiation” refers to the development of a cell from a more primitive stage towards a more mature (i.e., less primitive) cell, typically exhibiting phenotypic features of commitment to a particular cellular lineage. For generation of endothelial cells from the pluripotent stem cells, the stem cells are first differentiated to mesoderm commitment.

As used herein, a “early mesoderm progenitor” refers to a cell that is more differentiated than a pluripotent stem cell and which is committed to the mesodermal lineage. As described herein, an early mesoderm progenitor can be obtained from a PSC by culture with an agent that activates Wnt signaling, such as a GSK-3β inhibitor (e.g., CHIR99021 for two days).

In embodiments, cells can be identified and characterized based on expression of one or more biomarkers, such as particular biomarkers of early mesoderm progenitors or of differentiated endothelial cells. Non-limiting examples of biomarkers whose expression can be assessed in the characterization of cells of interest include CD31, VE−cadherin, KDR, vWF, FL1, and/or CD34 as biomarkers for differentiated endothelial cells.

### II. Culture Media Components

The method of the disclosure for generating endothelial cells from pluripotent stem cells comprise culturing early mesoderm progenitors in a culture media comprising specific agonist and/or antagonists of cellular receptors and/or signaling pathways to generate differentiated ECs. Moreover, the early mesoderm progenitors first can be obtained from pluripotent stem cells (e.g., ESCs or iPSCs) by culture of the stem cells under defined culture conditions as described herein.

A culture media comprising a VEGFR agonist, an FGFR agonist, a retinoic acid agonist, a sonic hedgehog antagonist, heparin or heparin mimetic and a Wnt pathway antagonist is sufficient to generate CD31+KDR+FL1+VE−cadherin+ endothelial cells from early mesoderm progenitor cells in as little three days of culture. The early mesoderm progenitors can be obtained from PSCs by culture of the PSCs with a Wnt pathway agonist for two days, leading to an overall five day protocol to obtain differentiated ECs from PSCs under defined culture conditions.

As used herein, an “agonist” of a cellular receptor or signaling pathway is intended to refer to an agent that stimulates (upregulates) the cellular receptor or signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

Agonists and antagonists used in the chemically-defined media and methods of the disclosure are known in the art and commercially available. They are used in the culture media at a concentration effective to achieve the desired outcome, e.g., generation of cells of interest (such as early mesoderm progenitors or differentiated endothelial cells) expressing markers of interest. Non-limiting examples of suitable agonist and antagonists agents, and effective concentration ranges, are described further below.

Agonists of the VEGFR pathway include agents, molecules, compounds, or substances capable of stimulating (upregulating) the vascular endothelial growth factor receptor signaling pathway, which biologically is activated by binding of VEGF to VEGFR. In an embodiment, the VEGFR agonist is VEGF or an analog thereof that stimulates signaling through VEGR. In an embodiment, the VEGFR agonist is VEGF (e.g., recombinant human VEGF). In an embodiment, the VEGFR agonist is VEGF, which is present in the culture media at a concentration within a range of 10-50 ng/ml, 15-45 ng/ml, 20-40 ng/ml, 20-30 ng/ml or at a concentration of 25 ng/ml.

Agonists of the FGFR pathway include agents, molecules, compounds, or substances capable of stimulating (upregulating) a fibroblast growth factor receptor signaling pathway, which biologically is activated by binding of an FGF to an FGFR. In an embodiment, the FGFR agonist is FGF2, SUN11602, or combinations thereof. In an embodiment, the FGFR pathway agonist is present in the culture media at a concentration within a range of 1-20 ng/ml, 5-15 ng/ml, 7.5-12.5 ng/ml, 9-11 ng/ml or at a concentration of 10 ng/ml. In an embodiment, the FGFR agonist is FGF2 (e.g., recombinant human FGF2). In an embodiment, the FGFR agonist is FGF2 which is present in the culture media at a concentration within a range of 1-20 ng/ml, 5-ng/ml, 7.5-12.5 ng/ml, 9-11 ng/ml or at a concentration of 10 ng/ml.

Agonists of the RA pathway include agents, molecules, compounds, or substances capable of stimulation of a retinoic acid receptor (RAR) that is activated by both all-trans retinoic acid and 9-cis retinoic acid. There are three RARs: RAR-alpha, RAR-beta and RAR-gamma, which are encoded by the RARA, RARB, RARG genes, respectively. Different retinoic acid analogs have been synthesized that can activate the retinoic acid pathway. Non-limiting examples of such compounds include TTNPB (agonist of RAR-alpha, beta and gamma), AM 580 (RARalpha agonist), CD 1530 (potent and selective RARgamma agonist), CD 2314 (selective RARbeta agonist), Ch 55 (potent RAR agonist), BMS 753 (RARalpha-selective agonist), Tazarotene (receptor-selective retinoid; binds RAR-beta and -gamma), Isotretinoin (endogenous agonist for retinoic acid receptors; inducer of neuronal differentiation), and AC 261066 (RARβ2 agonist). In some embodiments, the RA signaling pathway agonist is selected from the group consisting of: i) a retinoid compound, ii) a retinoid X receptor (RXR) agonist, and iii) a 25 retinoic acid receptor (RARs) agonist. In particular embodiments, the RA pathway agonist is selected from the group consisting of: retinoic acid, Sr11237, adapalene, EC23, 9-cis retinoic acid, 13-cis retinoic acid, 4-oxo retinoic acid, and All-trans Retinoic Acid (ATRA).

Accordingly, in one embodiment, the RA pathway agonist is selected from the group consisting of TTNPB, AM 580, CD 1530, CD 2314, CD 437, Ch 55, BMS 753, BMS 961, Tazarotene, Tamibarotene, Isotretinoin, Tretinoin, AC 261066, AC 55649, retinoic acid (RA), Sr11237, adapalene, EC23, 9-cis retinoic acid, 13-cis retinoic acid, 4-oxo retinoic acid, and All-trans Retinoic Acid (ATRA), AY 9944 dihydrochloride, Ciliobrevin A, Cyclopamine, or combinations thereof. In one embodiment, the RA pathway agonist is present in the culture media at a concentration within a range of 10-100 nM, 20-80 nM, 25-75 nM or 40-60 nM, or at a concentration of 50 nM. In one embodiment, the RA pathway agonist is TTNPB. In one embodiment, the RA pathway agonist is TTNPB, which is present in the culture media at a concentration within a range of 10-100 nM, 20-80 nM, 25-75 nM or 40-60 nM. In one embodiment, the RA pathway agonist is TTNPB, which is present in the culture media at a concentration of 50 nM.

Antagonists of the SHH (sonic hedgehog) pathway include agents, molecules, compounds, or substances capable of inhibiting (downregulating) signaling through the SHH pathway, which biologically involves binding of SHH to the Patched-1 (PTCH1) receptor and transduction through the Smoothened (SMO) transmembrane protein. In one embodiment, the SHH pathway antagonist is selected from the group consisting of Sant-1, Saikosaponin B1, Itraconazole, GANT61, MK4101, HPI-4, Vismodegib, Jervine, JK184, Taladegib, Ciliobrevin D, Dynapyrazole A, Dynarrestin, GANT58, HPI1, IHR1, PF 04449913 maleate, SANT-2, U 18666A, and combinations thereof. In one embodiment, the SHH pathway antagonist is present in the culture media at a concentration within a range of 10-100 nM, 20-80 nM, 25-75 nM or 40-nM or at a concentration of 50 nM. In one embodiment, the SHH pathway antagonist is Sant-1. In one embodiment, the SHH pathway antagonist is Sant-1, which is present in the culture media at a concentration of 10-100 nM, 20-80 nM, 25-75 nM or 40-60 nM. In one embodiment, the SHH pathway antagonist is Sant-1, which is present in the culture media at a concentration of nM.

Heparin is a glycosaminoglycan anticoagulant long known in the art and heparin mimetics are synthetic and semi-synthetic compounds that are highly sulfated, structurally distinct analogues of glycosaminoglycans. In embodiments, the culture media comprises heparin or a heparin mimetic, such as a heparin analogs selected from the group consisting of heparan sulfate, enoxaparin, small molecular weight heparins, AV5026, M402, and combinations thereof. In one embodiment, heparin or heparin mimetic is present in the culture media at a concentration within a range of 5-50 ng/ml, 10-40 ng/1, 15-25 ng/ml or 25-35 ng/ml, or at a concentration of 20 ng/ml. In one embodiment, the media comprise heparin, which is present in the culture media at a concentration within a range of 5-50 ng/ml, 10-40 ng/1, 15-25 ng/ml or 25-35 ng/ml. In one embodiment, the media comprise heparin, which is present in the culture media at a concentration of 20 ng/ml.

Antagonists of the WNT pathway include agents, molecules, compounds, or substances capable of inhibiting (downregulating) the canonical Wnt/β-catenin signaling pathway, which biologically is activated by binding of a Wnt-protein ligand to a Frizzled family receptor. In one embodiment, the WNT pathway antagonist is selected from the group consisting of XAV939, ICG-001 (Foscenvivint), Capmatinib (INCB28060), endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, iCRT14, WIF-1, DKK1, Isoquercitrin, Lanatoside C, Gigantol, RCM-1, WIKI4, IQ-1, Adavivant, PRI-724, Tegatrabetan, and combinations thereof. In one embodiment, the WNT pathway antagonist is present in the culture media at a concentration within a range of 10-500 nM, 50-250 nM, 50-150 nM, 75-125 nM or at a concentration of 100 nM. In one embodiment, the WNT pathway antagonist is XAV939. In one embodiment, the WNT pathway antagonist is XAV939, which is present in the culture media at a concentration of 10-500 nM, 50-250 nM, 50-150 nM or 75-125 nM. In one embodiment, the WNT pathway antagonist is XAV939, which is present in the culture media at a concentration of 100 nM.

Agonists of the WNT pathway include agents, molecules, compounds, or substances capable of stimulating (upregulating) the canonical Wnt/β-catenin signaling pathway, which biologically is activated by binding of a Wnt-protein ligand to a Frizzled family receptor. In one embodiment, a WNT pathway agonist is a glycogen synthase kinase 3 (Gsk3) inhibitor. In one embodiment, the WNT pathway agonist is selected from the group consisting of CHIR99021, CHIR98014, SB 216763, SB 415286, LY2090314, 3F8, A 1070722, AR-A 014418, BIO, BIO-acetoxime, AZD1080, WNT3A, alsterpaullone, indirubin-3-oxime, 1-azakenpaullone, kenpaullone, TC-G 24, TDZD 8, TWS 119, NP 031112, AT 7519, KY 19382, AZD2858, and combinations thereof. In one embodiment, the WNT pathway agonist is present in the culture media at a concentration within a range of 3.0-9.0 μM, 4.0-8.0 μM, 5.0-7.0 μM, 6.5-7.5 μM or at a concentration of 6.0 μM. In one embodiment, the WNT pathway agonist is CHIR99021. In one embodiment, the WNT pathway agonist is CHIR99021, which is present in the culture media at a concentration within a range of 3.0-9.0 μM, 4.0-8.0 μM, 5.0-7.0 μM or 6.5-7.5 μM. In one embodiment, the WNT pathway agonist is CHIR99021, which is present in the culture media at a concentration of 6.0 μM (e.g., in the stage 1 culture media for generating early mesoderm progenitors).

### III. Culture Conditions

In combination with the chemically-defined and optimized culture media described in subsection II above, the methods of generating endothelial cells of the disclosure utilize standard culture conditions established in the art for cell culture. For example, cells can be cultured at 37° C. and under 5% CO₂ conditions. Cells can be cultured in standard culture vessels or plates, such as culture dishes, culture flasks or 96-well plates. Pluripotent stem cells can be cultured in commercially-available media prior to differentiation. For example, stem cells can be cultured for at least one day in in a specialized stem cell media such as Essential 8 Flex media (Thermo Fisher #A2858501) prior to the start of the differentiation protocol. In a non-limiting exemplary embodiment, stem cells are passaged onto vitronectin (Thermo Fisher #A14700) coated 96-well plates at 150,000 cells/cm2 density and cultured for one day in Essential 8 Flex media prior to differentiation.

To begin the differentiation protocol starting from stem cells, the media the stem cells are being cultured in is changed to a basal differentiation media that has been supplemented with a Wnt signaling pathway agonists, such as a GSK-3β inhibitor (e.g., CHIR99021) as described above in subsection II, referred to herein as stage 1 of the differentiation protocol. In an embodiment, cells are grown in suspension, such as in a vertical wheel bioreactor during the differentiation process. For the differentiation, a basal differentiation media can include, for example, a commercially-available base supplemented with additional standard culture media components needed to maintain cell viability and growth, but typically lacking serum (the basal differentiation media is a serum-free media). Non-limiting examples of a commercially available base media include IMDM and F-12 media. A non-limiting example of a basal differentiation media is shown in Table 1:

TABLE 1
Sample Basal Differentiation Media

| Compound | Final | |
|---|---|---|
| Albumax | 0.25% | |
| Ethanolamine | 10 | uM |
| Monothioglycerol | 150 | uM |
| Cholesterol Sulfate | 200 ng/mL | (409 nM) |
| Trolox | 10 | uM |
| Linoleic Acid | 4 ng/mL | (0.01426 mM) |
| Oleic Acid | 10 | uM |
| Insulin | 10 | ug/mL |
| Holo-transferrin | 10 | ug/mL |
| Sodium Selenite | 5 | ng/mL |
| Vit C Phosphate | 100 | ug/mL |

| | |
|---|---|
| IMDM:F12 | 50:50 |

In certain embodiments, the starting pluripotent stem cells are adhered to plates, preferably coated with an extracellular matrix material such as vitronectin. In one embodiment, the stem cells are cultured on a vitronectin coated culture surface (e.g., vitronectin coated 96-well plates).

The culture media typically is changed regularly to fresh media. For example, in one embodiment, media is changed every 24 hours.

To generate early mesoderm progenitors, the starting pluripotent stem cells are cultured in a media comprising a GSK-3β inhibitor (e.g., CHIR99021) for sufficient time for cellular differentiation and expression of committed early mesoderm progenitor-associated markers, typically two days. For this stage, cells can be cultured in adherent or suspension conditions. This protocol for generation of early mesoderm progenitors from PSCs is referred to herein as “step (a)” or “stage 1.”

To generate differentiated endothelial cells from the early mesoderm progenitors, the progenitors are cultured, typically in suspension conditions, in a media comprising a VEGFR agonist, an FGFR agonist, a retinoic acid receptor agonist, a sonic hedgehog antagonist, heparin or heparin mimetic and a Wnt pathway antagonist for sufficient time for cellular differentiation and expression of endothelial cell-associated markers, typically three days. This protocol for generation of endothelial cells from early mesoderm progenitors is referred to herein as “step (b)” or “stage 2.”

In various embodiments, the early mesoderm progenitors are cultured in the optimized culture media for sufficient time to increase the expression of at least one, and preferably a plurality of, endothelial cell-associated markers. Non-limiting examples of suitable EC-associated markers include CD31, KDR, FL1, vWF, VE−cadherin and CD34. In embodiments, cells are cultured for sufficient time to increase the expression levels of at least two, at least three, at least four or at least five EC-associated markers. In an embodiment, cells are cultured for sufficient time to increase the expression level of at least one EC-associated marker by at least 10%, 20%, 30%, 40%, 50%, 60%, 70%, 80%, 90% or 100% as compared to the starting cell population. The level of expression of markers in the cultured ECs can be measured by techniques available in the art (e.g., qPCR analysis and/or flow cytometry).

Accordingly, in the first stage of the method, which generates early mesoderm progenitors from PSCs, pluripotent stem cells are cultured in the stage 1-optimized culture media on days 0-2, or starting on day 0 and continuing through day 2, or for 48 hours (2 days), or for at least 36 hours, or at least 40 hours, or at least 44 hours, or at least 48 hours. Accordingly, in the second stage of the method, which generates endothelial cells from early mesoderm progenitors, the early mesoderm progenitors generated in stage 1 are further cultured in the stage 2-optimized culture media on days 2-5, or starting on day 2 and continuing through day 5, or starting on day 2 and continuing for 72 hours (3 days), or starting on day 2 and continuing for at least 60 hours, or at least 64 hours, or at least 68 hours, or at least 70 hours, or at least 72 hours, or starting on day 2 and continuing for 60 hours, or for 64 hours, or for 68 hours, or for 70 hours or for 72 hours.

The culture media typically is changed regularly to fresh media. For example, in certain embodiments, media is changed every 24 hours, or every 48 hours or every 72 hours.

### IV. Uses

The methods and compositions of the disclosure for generating endothelial cells allow for efficient and robust availability of these cell populations for a variety of uses. For example, the methods and compositions can be used in the study of EC development and differentiation, including biology to assist in the understanding of vascular diseases and disorders.

The ECs obtained according to the methods of the disclosure can be further cultured in standard endothelial cell culture media, such as for two or more days, leading to them becoming functional endothelial cells as measured by standard assays demonstrating endothelial cell function. Non-limiting examples of standard assays demonstrating endothelial cell function include tube formation assays, ac-LDL assays and/or nitric oxide production assays.

Thus, the ECs obtained according to the methods of the disclosure offer the opportunity to investigate functional endothelial assembly. Other uses include for 3D-bioprinting, drug screening, safety assessments, organ on a chip, vascular tissue engineering and disease modeling.

The culture methods and compositions of the disclosure have been validated to work on bioreactors, thereby allowing for obtention of large numbers of endothelial cells from pluripotent stem cells, which is important for consistency and low variability.

ECs generated according to the methods of the disclosure also are contemplated for use in the treatment of various vascular diseases and disorders, for example either through delivery of the cells to a subject having the disease or disorder or use of the cells ex vivo to assemble vascular elements that are then delivered to a subject. Examples of vascular diseases and disorders include, but are not limited to, ischemic cardiovascular disease, peripheral vascular disease and blood vessel damage due to infection with SARS-CoV-2. Contemplated therapeutic uses include for therapeutic neovascularization, for enhancing blood perfusion and for promoting tissue repair.

### V. Compositions

In other aspects, the disclosure provides compositions related to the methods of generating endothelial cells, including culture media and isolated cell cultures.

In one aspect, the disclosure provides a culture media for generating CD31+ endothelial cells (ECs) comprising a VEGFR agonist, an FGFR agonist, a retinoic acid (RA) pathway agonist, a sonic hedgehog (SHH) antagonist, heparin or heparin mimetic and a WNT pathway antagonist. Non-limiting examples of suitable agents, and concentrations therefor, include those described in subsection II above. In an embodiment, the VEGFR agonist is VEGF, the FGFR agonist is FGF2, the RA pathway agonist is TTNBP, the SHH antagonist is Sant-1, the heparin or heparin mimetic is heparin and the WNT pathway antagonist is XAV939. In an embodiment, VEGF is at a concentration of 25 ng/ml, FGF2 is at a concentration of 10 ng/ml, TTNBP is at a concentration of 50 nM, Sant-1 is at a concentration of 50 nM, heparin is at a concentration of 20 ng/ml and XAV939 is at a concentration of 100 nM.

In another aspect, the disclosure provides an isolated cell culture of human CD31+ endothelial cells (ECs), the culture comprising: human CD31+ ECs cultured in a culture media comprising a VEGFR agonist, an FGFR agonist, a retinoic acid (RA) pathway agonist, a sonic hedgehog (SHH) antagonist, heparin or heparin mimetic and a WNT pathway antagonist. Non-limiting examples of suitable agents, and concentrations therefor, include those described in subsection II above. In an embodiment, the VEGFR agonist is VEGF, the FGFR agonist is FGF2, the RA pathway agonist is TTNBP, the SHH antagonist is Sant-1, the heparin or heparin mimetic is heparin and the WNT pathway antagonist is XAV939. In an embodiment, VEGF is at a concentration of 25 ng/ml, FGF2 is at a concentration of 10 ng/ml, TTNBP is at a concentration of 50 nM, Sant-1 is at a concentration of 50 nM, heparin is at a concentration of 20 ng/ml and XAV939 is at a concentration of 100 nM.

The present invention is further illustrated by the following examples, which should not be construed as further limiting. The contents of figures and all references, patents and published patent applications cited throughout this application are expressly incorporated herein by reference.

### EXAMPLES

### Example 1: Protocol Development for the Generation of iPSC-Derived Endothelial Cells

A two-stage recipe for generation of endothelial cells was developed that can guide human IPSC to endothelial cells expressing CD31, VE−cadherin, CD34, FlI1 and KDR after 5 days in culture.

First, undifferentiated iPSC were grown for 48 h in CDM2 media containing 6 uM CHIR99021 (referred to herein as stage 1). Then stage 2 media was engineered. The basis of this was an 8-factor HD-DoE experiment with focus on differentiation of cells toward the endothelial lineage for an additional 3 days after termination of stage 1 treatment. To test the effectors, 48 different combinations of effectors generated using Design-of-Experiments compression through D-optimality were robotically prepared. The effector combinations were prepared in a basal media and were subsequently added to the cells, which were then allowed to differentiate. Three days later, RNA extraction was performed, and gene expression was obtained using quantitative PCR analysis. The data were normalized and modeled using partial least squares regression analysis to the effector design, resulting in the generation of gene-specific models, which after model tuning for maximal Q2 predictive power, provided explanation of the effectors ability to control the expression of individual genes, combinatorically, and individually. Solutions within the tested space could then be explored to address desirability. Optimizing for maximal expression of CD31 led to a robust solution. At this solution, other genes were also predicted to be abundantly expressed, such as CD34, ERG, LMO2, FLI1, TAL1 and CDH5, all genes highly expressed on endothelial cells, suggesting cell commitment to this lineage. On this model, we observed high levels of KDR, important receptor for VEGF signaling. On the same model, NKX2-5, OTX2 and GBX2 were downregulated (gene associated to other lineages, such as heart and brain).

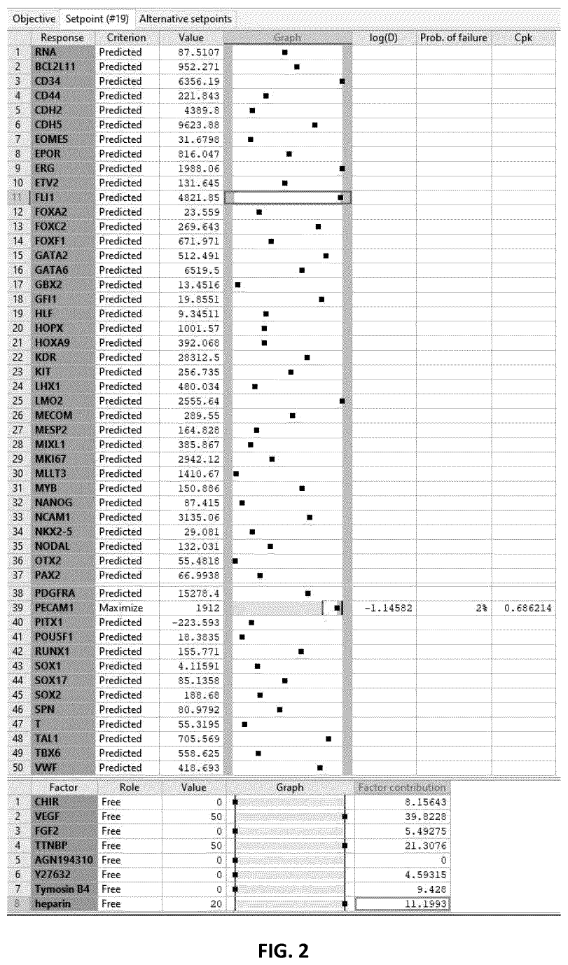

Additionally, genes related to the earlier stages such as, T, NODAL and EOMES, were downregulated, indicating that cells are differentiating. This model was derived from initial testing of eight factors including: VEGF, FGF2, TTNBP, AGN194310, Y27632, Thymosin B4 and Heparin. Three of these effectors: VEGF, TTNBP and heparin, showed positive impact on expression of genes of interest with 39, 21 and 11 factor contributions, respectively (FIG. 2). Within the specifications of attaining approximate 85% maximal expression of PECAM, this complex media composition had a Cpk value (process capability index) of 0.68, with a corresponding 2% risk of failure.

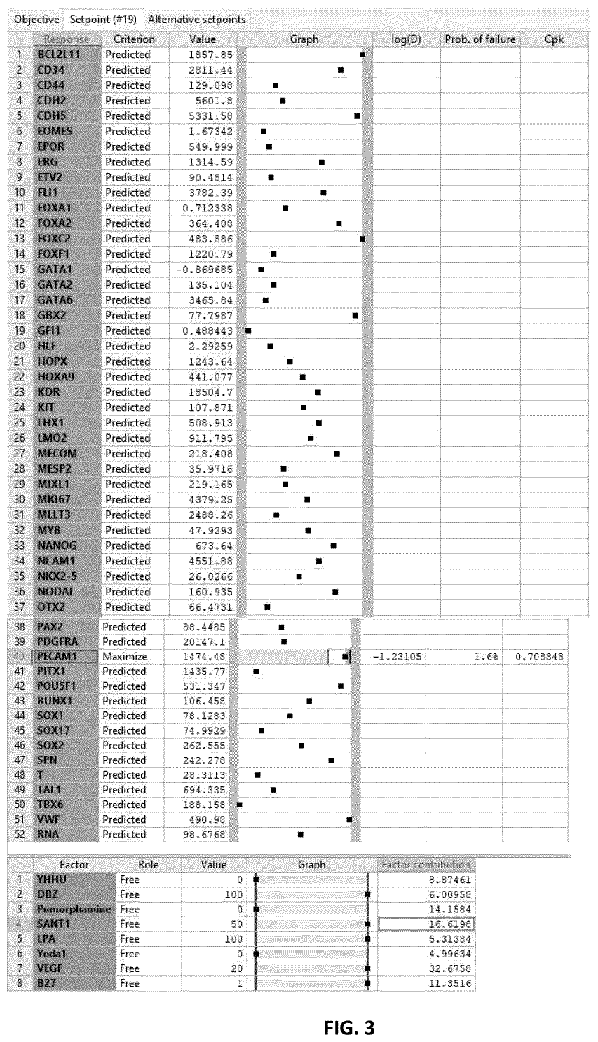

Next, we evaluated further factors to possibly increase the complexity of the signaling inputs to attain effective fate control. As previously, we focused on expression of CD31. Optimizing for maximal expression of the CD31 led to a robust solution. At this solution, other genes were also predicted to be abundantly expressed, such as CD34, CDH5, vWF, FOXC2, all genes related to the endothelial program suggesting that cells are commitment to this lineage. This model was derived from initial testing of eight factors including: YHHU-3792, DBZ, Pumorphamine, SANT1, LPA, YODA1, VEGF and B27. Two of these effectors: VEGF and SANT1, showed positive impact on expression of genes of interest with high factor contribution of 32, and 16 factor contributions, respectively (FIG. 3). We did not include B27 (a commonly used additive to enrich basal media for neural or endothelial cells) in our recipe, since it is a complex mixture and expensive, and we got sufficient induction of CD31 without and using our differentiation factors together with our basal media. DBZ and LPA had low contributions factors and therefore were not used in the recipe.

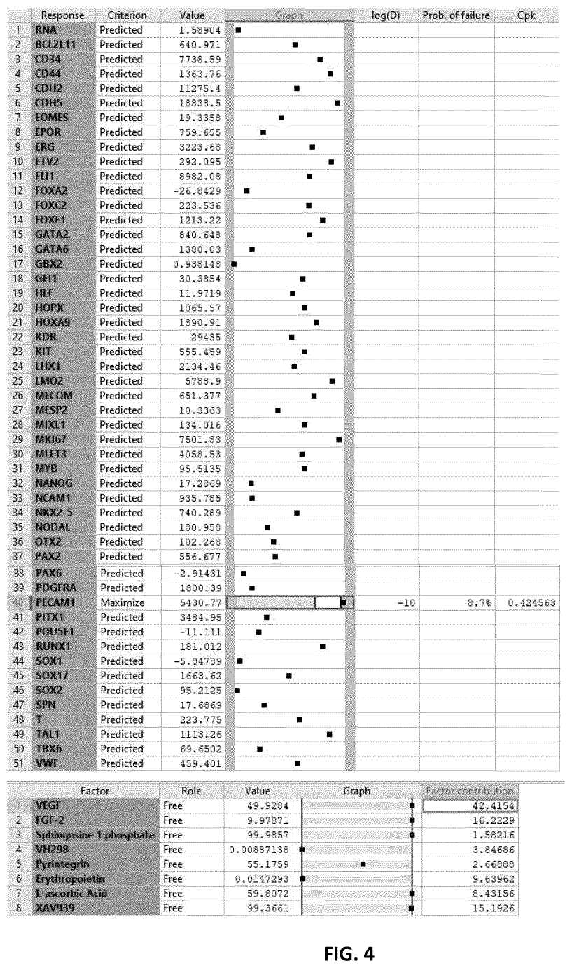

To further improve the recipe for endothelial cell differentiation, we performed additional HD-DoE experiments. This model was derived from initial testing of eight factors including: VEGF, FGF2, Sphingosine 1 phosphate, VH298, Pyrintegrin, Erythropoetin, L-ascorbic acid and XAV939. Three of these effectors: VEGF, FGF2 and XAV939 showed positive impact on expression of genes of interest with 42, 16 and 15 factor contributions, respectively (FIG. 4). At this solution, other genes were also predicted to be abundantly expressed, such as CD34, CD44, ETV2, MKI67, LMO2, FLI1, ERG, TAL1 and CDH5, all genes highly expressed on endothelial cells, suggesting cell commitment to this lineage. On this model, we observed high levels of KDR, important receptor for VEGF signaling. Because FGF2 was critical on this model for expression of endothelial genes, and on the first model did not significantly impact the expression of CD31, we decided to keep FGF2 in the recipe. Considering all the models analyzed, based on predicted conditions that maximize expression of endothelial enriched genes such as ERG, CDH5, CD31, FLI1, CD34, a complex recipe for endothelial differentiation was developed that is composed of 6 effectors, as shown below in Table 2:

TABLE 2
Validated effectors for endothelial differentiation stage 2 recipe

| Agent | Role | Concentration | |
|---|---|---|---|
| VEGF | VEGFR agonist | 25 | ng/mL |
| FGF2 | FGFR agonist | 10 | ng/mL |
| TTNBP | RA pathway agonist | 50 | nM |
| Sant1 | SHH antagonist | 50 | nM |
| Heparin | Glycosaminoglycan | 20 | ng/mL |
| XAV939 | WNT pathway antagonist | 100 | nM |

### Example 2: Factor Criticality Analysis of Endothelial Cell-Inducing Culture Conditions

The various contribution factors for each protocol input for the culture inducing endothelial cells suggested variable relative influence. To assess the impact of elimination of each validated factor on genes associated with the endothelial lineage, dynamic profile analysis was used and the expression levels of genes of interest were compared in the absence of each finalized factor while others are kept present. Since expression levels of genes of interest reveal whether the desired outcome is reachable, this factor criticality analysis revealed the extent of importance of each input effector.

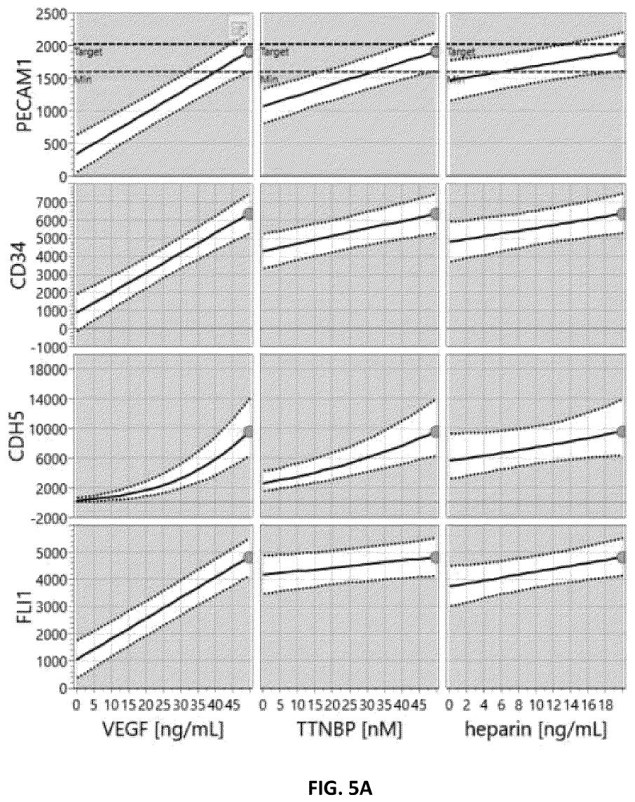

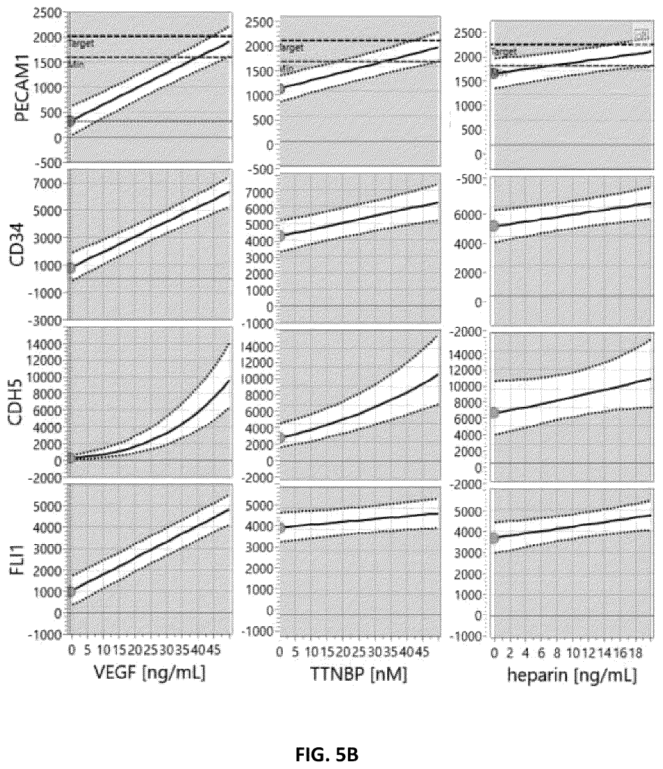

In this analysis, removal of a finalized effector while keeping other factors was used to analyze the impact of factors on the expression levels of CD31, CD34, FLi1 and CDH5 (FIG. 5A). When VEGF was removed, values of CD31 decreased from 2000 to 340, values of CD34 changed from 6500 to 1000, values of CDH5 decreased from 10000 to 0 and values of FLI1 dropped from 4900 to 1035. All changes represent a significant loss of expression of a desired gene (FIG. 5B). When TTNBP was removed, values of CD31 decreased from 2000 to 1077, values of CD34 decreased from 6500 to 4300, values of CDH5 decreased from 10000 to 2600 and values of Fli1 decreased from 2883 to 1703. TTNBP removal did not affect Fli1 expression significantly (FIG. 5B). When heparin was removed, values of CD31 decreased from 2000 to 1500, values of CD34 decreased from 6500 to 4800, values of CDH5 decreased from 10000 to 5800 and values of Fli1 decreased from 2883 to 1703. Heparin removal did not affect Fli1 expression significantly (FIG. 5B).

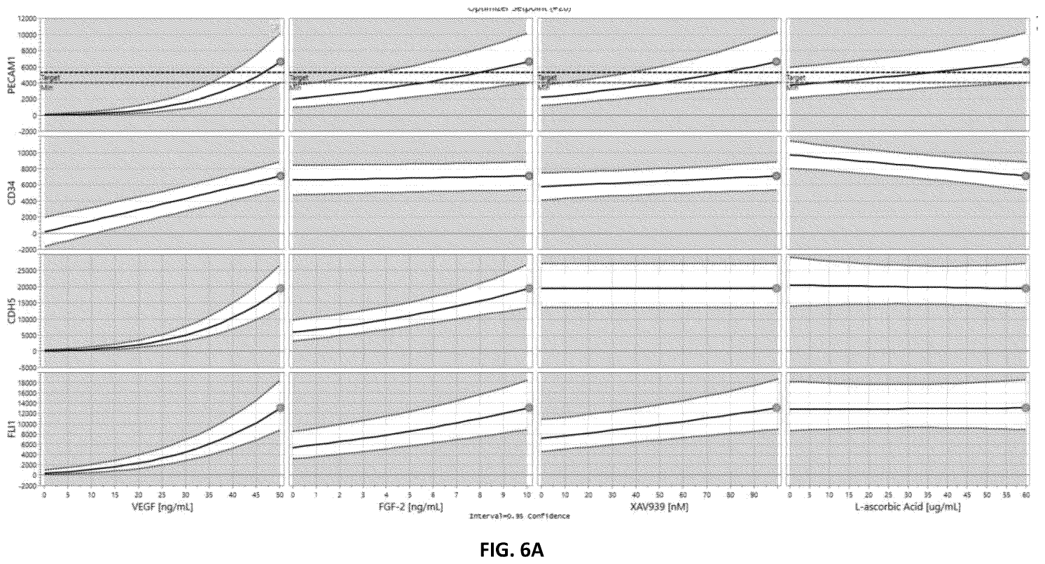

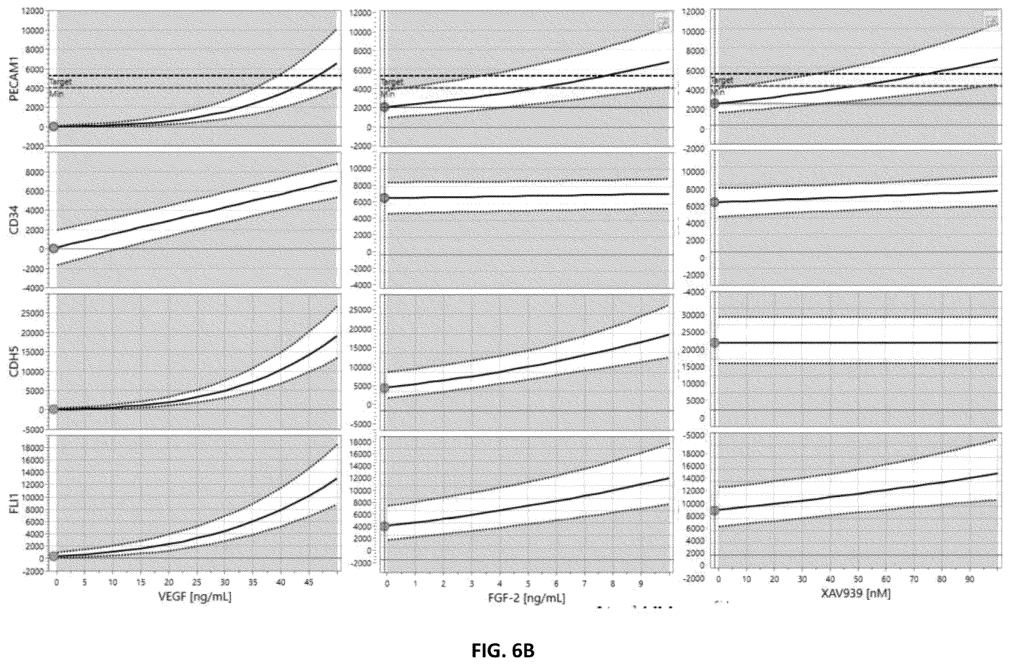

In another model, the effect of XAV939 and FGF2 on the expression levels of CD31, CD34, FLi1 and CDH5 was evaluated (FIG. 6A). Again, VEGF was critical for expression of all endothelial genes analyzed. When FGF2 was removed, values of CD31 decreased from 6500 to 1990, values of CD34 did not change significantly, values of CDH5 decreased from 19000 to 5000 and values of Fli1 dropped from 1300 to 500 (FIG. 6B). XAV939 removal decreased CD31 levels from 6500 to 2225, CD34 levels from 7180 to 5836 and Fli1 from 13400 to 7300.

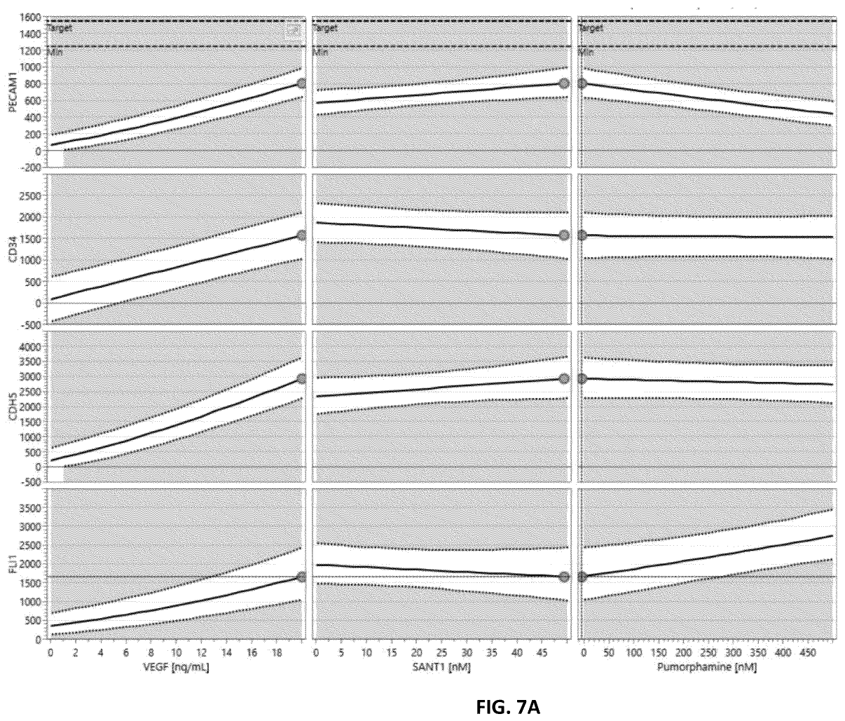

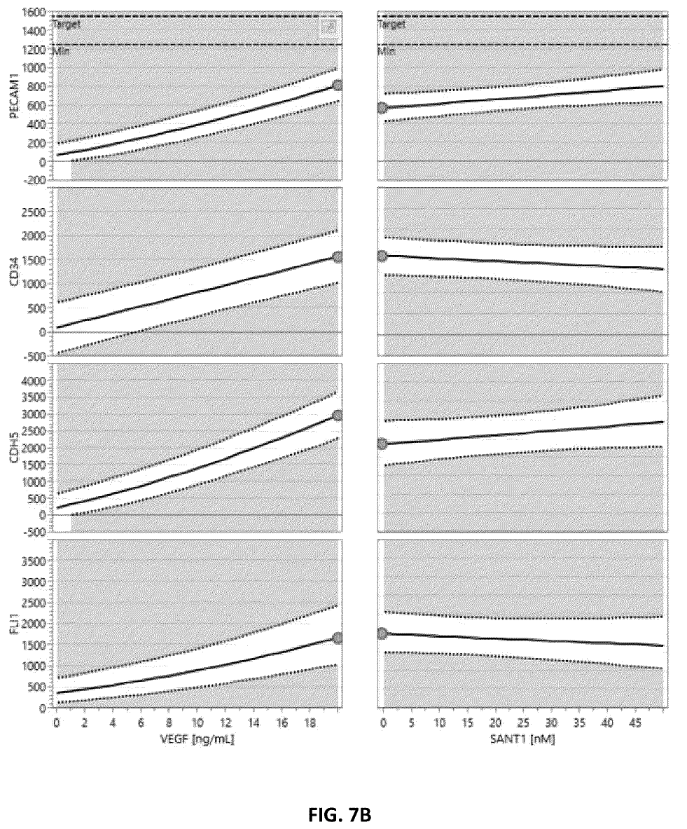

In another model, we evaluated the effect of SANT1, a sonic hedgehog (SHH) inhibitor, on the expression levels of CD31, CD34, FLi1 and CDH5 (FIG. 7A). When doing this dynamic profile analysis, L-ascorbic acid was kept in the model, since it is present in the basal media. When SANT1 was removed, CD31 levels went from 808 to 563 and CDH5 levels changed from 2928 to 2340 (FIG. 7B). Additionally, this model, showed how the SHH pathway is a critical regulator of the endothelial program. Addition of Pumorphamine, a SHH agonist, decreased CD31 levels significantly from 800 to 400, corroborating with the evidence that SANT1 is a good effector to be included in the stage 2 recipe.

### Example 3: Flow Cytometry Analysis and Immunocytochemistry of Stem Cell Derived Endothelial Cells Expressing Endothelial Cell Markers

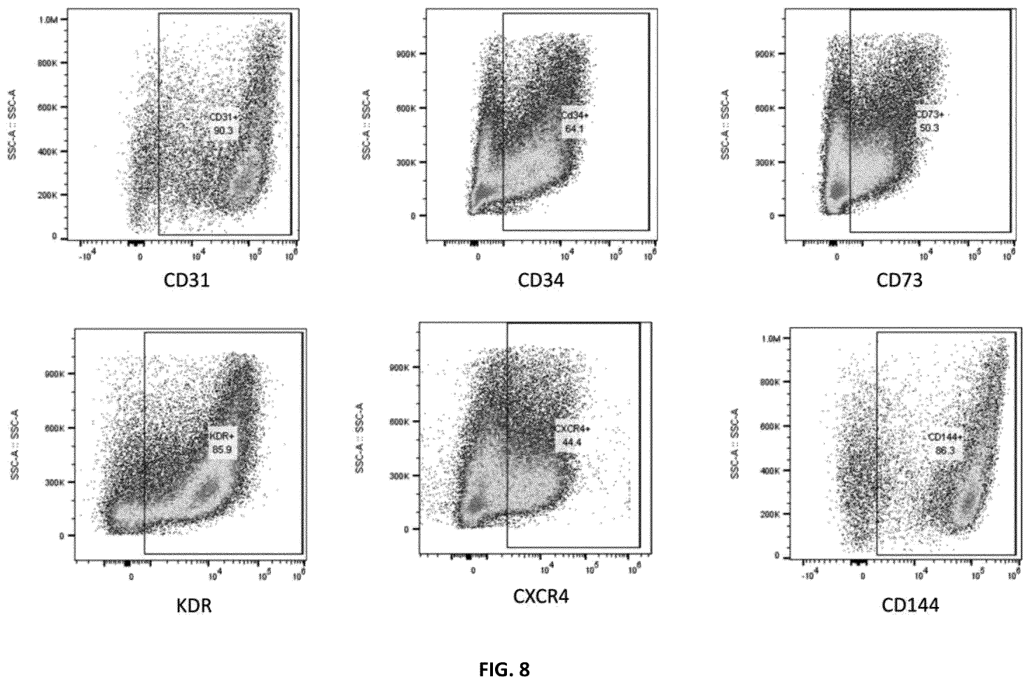

To further validate the developed recipe in Example 1, iPSC were grown for 2 days in stage 1 media, and then grown for 3 days in stage 2 media in a vertical wheel bioreactor, and flow cytometry and immunohistochemistry analysis was used to evaluate expression of endothelial markers. The basal differentiation media used for this experiment is shown in Table 1. Flow cytometry analysis confirmed the efficiency of the stage 2 recipe to promote conversion of iPSC to endothelial cells (FIG. 8). More than 80% of cells were positive for CD31, CD144 and KDR. Endothelial cells also expressed CD34, CXCR4 and CD73, however at a lower level.

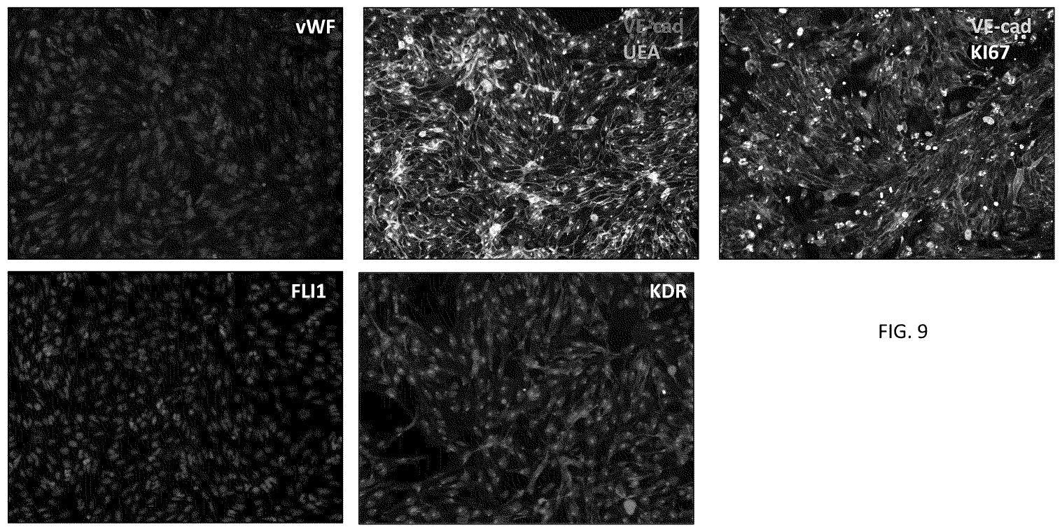

Additionally, immunofluorescence staining confirmed homogeneity and robust staining for various endothelial markers such as VE−cadherin, KDR, vWF and Fli1. Immunofluorescence staining showed that cells were in a proliferative state, as shown by KI67 staining. Finally, staining using Ulex europaeus Agglutinin I (UEA) (a lectin that has been established as a robust marker for endothelial cells) confirmed differentiation to the endothelial state (FIG. 9).

### Example 4: Functional Validation of iPSC-Derived Endothelial Cells

To evaluate the functionality of the iPSC-derived endothelial cells, three endothelial cell assays known in the art were performed: tube formation assay in a layer of Matrigel, acetylated LDL uptake, and nitric oxide measurement by using a probe called DAF-FM. Cells used for these assays were cryopreserved cells after stage 2. Cells were thawed in the basal media shown in Table 1 with the addition of 25 ng/mL of VEGF and 5 uM SB431542. All assays were conducted in the presence of the same media. Acetylated LDL and DAF-FM assays were conducted after cells recovering from thawing process (72-96 h). Tube formation assay was performed after passing cells once.

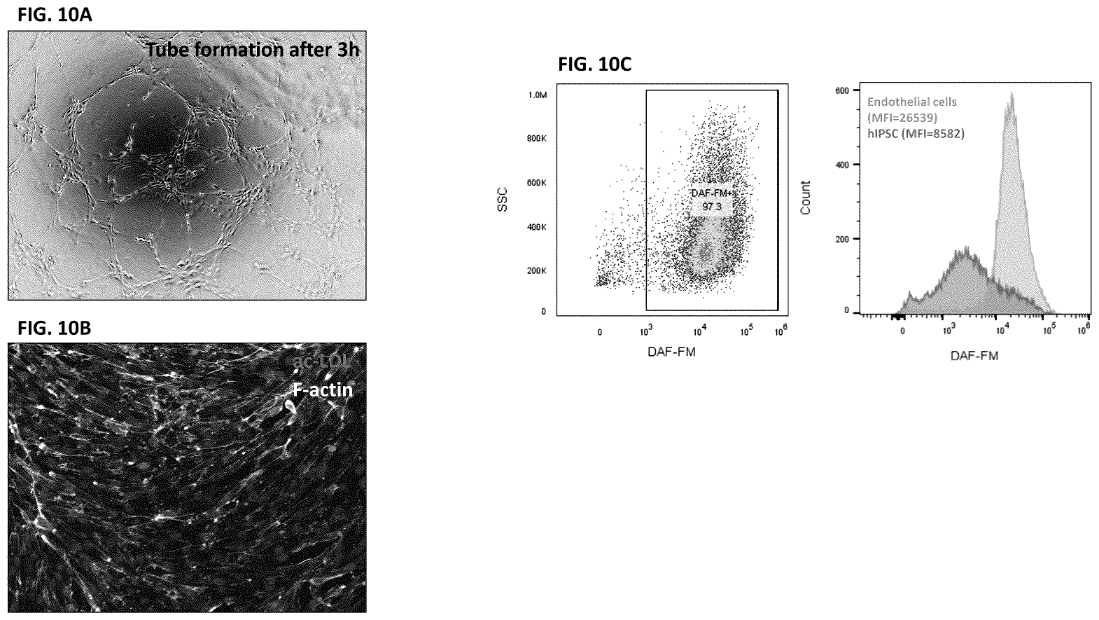

For the tube formation assay, 50K cells (in 100 uL of media) were plated on a layer of growth factor reduced Matrigel in a 96 well plate. Three hours later, pictures were taken to identify the formation of tube structures. Results are shown in FIG. 10A, which demonstrate the formation of tubes by the endothelial cells.

As background for the acetylated LDL assay, LDL containing an unmodified apoprotein is used to study normal cholesterol delivery and internalization. If the lysine residues of LDL's apoprotein have been acetylated, the LDL complex no longer binds to the LDL receptor, but rather is taken up by endothelial and microglial cells that possess “scavenger” receptors specific for that modified form. The results shown in FIG. 10B demonstrate that our iPSC-derived endothelial cells had the ability of uptake ac-LDL after 4 h incubation.

As background for the DAF-FM assay, DAF-FM is non-fluorescent until it reacts with nitric oxide forming a fluorescent compound called benzotriazole. DAF-FM staining showed that 97% of our endothelial cells are labelled indicating they are producing nitric oxide. As shown in FIG. 10C, when compared to undifferentiated iPSC, iPSC derived endothelial cells produce significant more nitric oxide than undifferentiated cells (mean fluorescence intensity, MFI=26539 versus MFI:8582 for undifferentiated cells).

### Example 5: RNA-Seq Analysis to Characterize iPSC-Derived Endothelial Cells

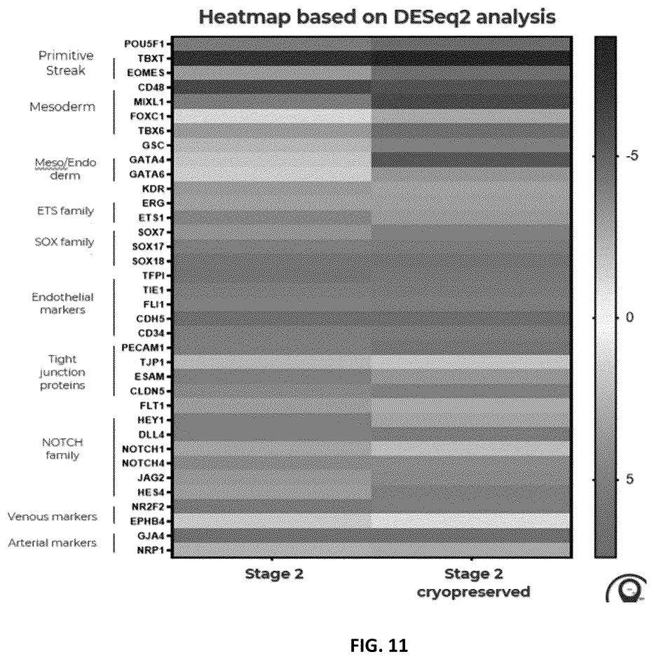

Bulk RNA-seq analysis was used to characterize iPSC-derived endothelial cells. Stage 1, stage 2, and cryopreserved stage 2 samples maintained for 4 days in culture were analyzed. Differential gene expression analysis was conducted comparing stage 1 samples to stage 2 samples. As shown in FIG. 11, transcriptome analysis revealed an early population of endothelial cells expressing both venous (NR2F2 and EPHB4) and arterial markers (GJA4 and NRP1). Expression of tight junction proteins (TJP1, PECAM1, CDLN5 ESAM), SOX transcription factors (SOX7, SOX17 and SOX18) and many NOTCH family members (HEY1, DLL4, JAG2, NOTCH1, NOTCH4) was observed. iPSC-derived endothelial cells expressed receptor tyrosine kinase for VEGFs, such as KDR and FLT1. Expression of endothelial specific ETS transcription factors (ETS1, ERG, FLI1) also was found.

Thus, RNA-seq characterization of endothelial cells confirmed upregulation of endothelial genes and downregulation of pluripotency, primitive streak and mesoderm genes. Overall, upregulation of desired genes and downregulation of stem cell markers confirms the conversion of iPSCs to endothelial cells in 5 days.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method of generating human CD31+ endothelial cells (ECs) comprising culturing human early mesoderm progenitor cells in a culture media comprising a VEGFR agonist, an FGFR agonist, a retinoic acid pathway agonist, a sonic hedgehog (SHH) antagonist, heparin or heparin mimetic and a WNT pathway antagonist to generate human CD31+ ECs.

2. The method of claim 1, wherein the early mesoderm progenitor cells are cultured for three days in the culture media.

3. The method of claim 1, wherein the early mesoderm progenitor cells are obtained by culturing human pluripotent stem cells in a media comprising a Wnt pathway agonist for two days.

4. A method of generating human CD31+ endothelial cells (ECs) comprising:
(a) culturing human pluripotent stem cells in a culture media comprising a Wnt pathway agonist on day 0-day2 to generate early mesoderm progenitor cells; and
(b) culturing the early mesoderm progenitor cells in a culture media comprising a VEGFR agonist, an FGFR agonist, a retinoic acid pathway agonist, a sonic hedgehog (SHH) antagonist, heparin or heparin mimetic and a WNT pathway antagonist on day 2-day 5 to generate CD31+ ECs.

5. The method of claim 4, wherein the ECs also express at least one marker selected from the group consisting of KDR, vWF, FL1, VE−cadherin and CD34.

6. The method of claim 4, wherein the Wnt pathway agonist is a GSK-3β inhibitor.

7. The method of claim 6, wherein the GSK-3β inhibitor is CHIR99021.

8. The method of claim 7, wherein CHIR99021 is present in the culture at a concentration in a range of 3.0-9.0 μM.

9. The method of claim 8, wherein CHIR99021 is present in the culture at a concentration of 6.0 μM.

10. The method of claim 4, wherein the VEGFR agonist is VEGF.

11. The method of claim 10, wherein VEGF is present in the culture at a concentration in a range of 10-50 ng/ml.

12. The method of claim 10, wherein VEGF is present in the culture at a concentration of 25 ng/ml.

13. The method of claim 4, wherein the FGFR agonist is FGF2 or SUN11602.

14. The method of claim 13, wherein the FGFR agonist is present in the culture at a concentration in a range of 1-20 ng/ml.

15. The method of claim 13, wherein the FGFR agonist is FGF2, which is present in the culture at a concentration in a range of 1-20 ng/ml.

16. The method of claim 13, wherein the FGFR agonist is FGF2, which is present in the culture at a concentration of 10 ng/ml.

17. The method of claim 4, wherein the retinoic acid (RA) pathway agonist is selected from the group consisting of TTNPB, AM 580, CD 1530, CD 2314, CD 437, Ch 55, BMS 753, BMS 961, Tazarotene, Tamibarotene, Isotretinoin, Tretinoin, AC 261066, AC 55649, retinoic acid (RA), Sr11237, adapalene, EC23, 9-cis retinoic acid, 13-cis retinoic acid, 4-oxo retinoic acid, and All-trans Retinoic Acid (ATRA), AY 9944 dihydrochloride, Ciliobrevin A, Cyclopamine, or combinations thereof.

18. The method of claim 17, wherein the RA pathway agonist is present in the culture at a concentration in a range of 10-100 nM.

19. The method of claim 17, wherein the RA pathway agonist is TTNPB, which is present in the culture at a concentration in a range of 25-75 nM.

20. The method of claim 17, wherein the RA pathway agonist is TTNPB, which is present in the culture at a concentration of 50 nM.

21. The method of claim 4, wherein the sonic hedgehog (SHH) antagonist is selected from the group consisting of Sant-1, Saikosaponin Bl, Itraconazole, GANT61, MK4101, HPI-4, Vismodegib, Jervine, JK184, Taladegib, Ciliobrevin D, Dynapyrazole A, Dynarrestin, GANT58, HPI1, IHR1, PF 04449913 maleate, SANT-2, U 18666A, and combinations thereof.

22. The method of claim 21, wherein the SHH antagonist is present in the culture at a concentration in a range of 10-100 nM.

23. The method of claim 21, wherein the SHH antagonist is Sant-1, which is present in the culture at a concentration in a range of 25-75 nM.

24. The method of claim 21, wherein the SHH antagonist is Sant-1, which is present in the culture at a concentration of 50 nM.

25. The method of claim 4, wherein heparin or heparin mimetic is selected from the group consisting of heparin, heparan sulfate, enoxaparin, small molecular weight heparins, AV5026, M402, and combinations thereof.

26. The method of claim 25, wherein heparin or heparin mimetic is present in the culture at a concentration in a range of 10-50 ng/ml.

27. The method of claim 25, wherein heparin which is present in the culture at a concentration in a range of 25-35 ng/ml.

28. The method of claim 25, wherein heparin is present in the culture at a concentration of 20 ng/ml.

29. The method of claim 4, wherein the Wnt pathway antagonist is selected from the group consisting of XAV939, ICG-001 (Foscenvivint), Capmatinib (INCB28060), endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, iCRT14, WIF-1, DKK1, Isoquercitrin, Lanatoside C, Gigantol, RCM-1, WIKI4, IQ-1, Adavivant, PRI-724, Tegatrabetan, or combinations thereof.

30. The method of claim 29, wherein the Wnt pathway antagonist is present in the culture at a concentration in a range of 10-500 nM.

31. The method of claim 29, wherein the Wnt pathway antagonist is XAV939, which is present in the culture at a concentration in a range of 50-150 nM.

32. The method of claim 29, wherein the Wnt pathway antagonist is XAV939, which is present in the culture at a concentration of 100 nM.

33. The method of claim 4, wherein the pluripotent stem cells are embryonic stem cells.

34. The method of claim 4, wherein the pluripotent stem cells are induced pluripotent stem cells.

35. A culture media for generating endothelial cells cells (ECs) comprising a VEGFR agonist, an FGFR agonist, a retinoic acid (RA) pathway agonist, a sonic hedgehog (SHH) antagonist, heparin or heparin mimetic and a WNT pathway antagonist.

36. The culture media of claim 35, wherein the VEGFR agonist is VEGF, the FGFR agonist is FGF2, the RA pathway agonist is TTNBP, the SHH antagonist is Sant-1, the heparin or heparin mimetic is heparin and the WNT pathway antagonist is XAV939.

37. The culture media of claim 36, wherein VEGF is at a concentration of 25 ng/ml, FGF2 is at a concentration of 10 ng/ml, TTNBP is at a concentration of 50 nM, Sant-1 is at a concentration of 50 nM, heparin is at a concentration of 20 ng/ml and XAV939 is at a concentration of 100 nM.

38. An isolated cell culture of human CD31+ endothelial cells, the culture comprising: human CD31+ endothelial cells cultured in a culture media comprising a VEGFR agonist, an FGFR agonist, a retinoic acid (RA) pathway agonist, a sonic hedgehog (SHH) antagonist, heparin or heparin mimetic and a WNT pathway antagonist.
